SEQUENCE LISTING (2 sequences)

SEQ ID NO: 1 - protein, 13 aa, Homo sapiens
  VARIANT (1)...(13): Xaa = Any Amino Acid
RXXLXXWXEXQXW

SEQ ID NO: 2 - protein, 851 aa, Homo sapiens
MAQWEMLQNLDSPFQDQLHQLYSHSLLPVDIRQYLAVWIEDQNWQEAALGSDDSKATMLFFHFLDQLNYECGRCSQDPESLLLQHNLRKFCRDIQPFSQDPTQLAEMIFNLLLEEKRILIQAQRAQLEQGEPVLETPVESQQHEIESRILDLRAMMEKLVKSISQLKDQQDVFCFRYKIQAKGKTPSLDPHQTKEQKILQETLNELDKRRKEVLDASKALLGRLTTLIELLLPKLEEWKAQQQKACIRAPIDHGLEQLETWFTAGAKLLFHLRQLLKELKGLSCLVSYQDDPLTKGVDLRNAQVTELLQRLLHRAFVVETQPCMPQTPHRPLILKTGSKFTVRTRLLVRLQEGNESLTVEVSIDRNPPQLQGFRKFNILTSNQKTLTPEKGQSQGLIWDFGYLTLVEQRSGGSGKGSNKGPLGVTEELHIISFTVKYTYQGLKQELKTDTLPVVIISNMNQLSIAWASVLWFNLLSPNLQNQQFFSNPPKAPWSLLGPALSWQFSSYVGRGLNSDQLSMLRNKLFGQNCRTEDPLLSWADFTKRESPPGKLPFWTWLDKILELVHDHLKDLWNDGRIMGFVSRSQERRLLKKTMSGTFLLRFSESSEGGITCSWVEHQDDDKVLIYSVQPYTKEVLQSLPLTEIIRHYQLLTEENIPENPLRFLYPRIPRDEAFGCYYQEKVNLQERRKYLKHRLIVVSNRQVDELQQPLELKPEPELESLELELGLVPEPELSLDLEPLLKAGLDLGPELESVLESTLEPVIEPTLCMVSQTVPEPDQGPVSQPVPEPDLPCDLRHLNTEPMEIFRNCVKIEEIMPNGDPLLAGQNTVDEVYVSRPSHFYTDGPLMPSDF

(19) United States
(12) Patent Application Publication
(10) Pub. No.: US 2004/0009571 A1
(43) Pub. Date: Jan. 15, 2004

(54) Dimerization interface of signal transducer and activator of transcription (STAT) proteins

(76) Inventors: John Kuriyan, Berkeley, CA (US); James E. Darnell, Larchmont, NY (US); Xiaomin Chen, Houston, TX (US); Focco Van den Akker, Cleveland, OH (US)

Correspondence Address: KLAUBER & JACKSON, 411 HACKENSACK AVENUE, HACKENSACK, NJ, 07601

(21) Appl. No.: 10/179,451

(22) Filed: Jun. 25, 2002

Related U.S. Application Data
(63) Continuation-in-part of application No. 10/045,792, filed on Oct. 19, 2001.
Application No. 10/045,792 is a division of application No. 09/556,273, filed on Apr. 24, 2000, now Pat. No. 6,312,887.
Application No. 09/556,273 is a division of application No. 09/012,710, filed on Jan. 23, 1998, now Pat. No. 6,087,478.

(51) Int. Cl. C12Q 1/68; G06F 19/00; G01N 33/48; G01N 33/50; C12N 9/22

(52) U.S. Cl. 435/199; 435/6; 702/20

(57) ABSTRACT

The invention identifies an interface domain for interaction between two or more dimers of Signal Transducer and Activator of Transcription (STAT) proteins formed between amino acid residues Gln8 (Q8), Ile12 (I12), and Leu15 (L15) of α helices 1 and 2, Met28 (M28) and Glu29 (E29) of α helix 3 of a first STAT protein partner of the dimer, and Leu77 (L77) and Leu78 (L78) in α helix 7 of a second STAT protein partner of the dimer. The interface domain is useful for designing and identifying compounds capable of enhancing or inhibiting binding between STAT protein dimers and/or DNA binding sites, and thus useful for identifying compounds able to modulate STAT protein dimer-dimer induction of gene expression.

### RELATED PATENT APPLICATIONS

This application is a continuation-in-part and claims priority under 35 USC §120 U.S. Ser. No. 10/045,792 filed Feb. 8, 2002, which is a divisional application of U.S. Ser. No. 09/556,273 filed Apr. 24, 2002, which is a divisional application of U.S. Ser. No. 09/012,710 filed Jan. 23, 1998, now U.S. Pat. No. 6,087,478, which applications are herein specifically incorporated by reference in their entirety.

### GOVERNMENT SUPPORT

The research leading to the present invention was supported, at least in part, by NIH Grant Nos. AI32489 and AI34420. Accordingly, the Government may have certain rights in the invention.

### FIELD OF THE INVENTION

The present invention relates generally to structural and functional properties of STAT proteins. More specifically, the present invention describes a physiologically relevant STAT dimer interface, and methods of using the structural information thereof, for example, in identifying potential therapeutic compounds capable of enhancing or inhibiting the interaction between STAT dimers.

### BACKGROUND OF THE INVENTION

The STAT (signal transducers and activators of transcription) proteins are a family of transcription factors involved in the activation of target genes in response to cytokines and growth factors (Darnell (1997) Science 277:1630-1635). The binding of these ligands to their cognate receptors leads to tyrosine kinase activation and phosphorylation of latent STAT monomers in the cytoplasm. Tyrosine phosphorylated STATs undergo homo- or hetero-dimerization via reciprocal SH2-phosphotyrosine interactions, followed by translocation to the nucleus and activation of gene expression. The canonical STAT recognition site on DNA is the palindromic sequence TTCN₃₋₄GAA. It has been shown that STAT1, STAT4 and STAT5 are able to form higher order complexes (dimer:dimer or higher) on promoters containing two or more neighbouring STAT binding sites (John et al. (1999) Mol. Cell. Biol. 19:1910-1918). This interaction between STAT dimers is cooperative, and is lost upon deletion of the N-domain of the STATs (Zhang and Darnell (2001) J. Biol. Chem. 276:33576-33581).

Earlier work with the STAT4 N-domain crystal structure (Vinkemeier et al. (1998) Science 279:1048-1052), involving mutation of amino acid residue Trp 37 (W37), located between to STAT molecules at a crystal packing interface, led to the loss of cooperative STAT binding to tandem sites on DNA (John et al. (1999) supra). Consequently, the physiologically relevant dimer-dimer interaction was interpreted as based on the interface domain containing Trp 37 (W37).

There is a need to obtain agonists and antagonists that can modulate the effect of STAT proteins during specific gene activation. In particular, there is a need to obtain drugs that will directly interact with the important N-terminal domain of STAT proteins. On method of screening for such compounds relies on structure based drug design, in which the three dimensional structure of a protein or protein fragment is determined and potential agonists and/or potential antagonists are designed with the aid of computer modeling (Bugg et al. (1993) Scientific American December: 92-98; West et al. (1995) TIPS 16:67-74).

### BRIEF SUMMARY OF THE INVENTION

The crystal structures of the N-terminal domain (N-domain) and the core region of the STAT family of transcription factors have been determined previously. STATs can form cooperative higher order structures (tetramers or higher oligomers) while bound to DNA.

From the crystal packing in the STAT4 N-domain crystal structure, determined at 1.5 Å resolution (Vinkemeier et al. (1998) Science 279:1048-1052), a dimer interface of the N-domains of STATs including Trp 37 (W37) was suggested (FIG. 1a, now termed “Interface I”). The experiments described herein, however, provide the results of site directed mutagenesis of residues predicted to be involved at a second dimer interface, shown in FIG. 1b and herein termed “Interface II”, including Phe 77 (F77) and Leu 78 (L78). Based on the results obtained upon mutation of amino acid residues Phe 77 and Leu 78 at one side of Interface II, an alternative model from that presented earlier (Vinkemeier et al. (1998) supra) for the N-domain dimer has been deduced.

In one aspect, the present invention provides a crystal of the N-terminal domain having a space group of P6₅22 and a unit cell of dimensions a=79.51 Å, b=79.51, and c=84.68 Å. The present invention further provides a crystal of the N-terminal domain having secondary structural elements comprising eight helices (α1-α8) that are assembled into a hook-like structure that has an inner and outer surface. The first four helices (α1-α4) form a ring-shaped element having a proximal and a distal surface, whereas helices six (α6) and seven (α7) form an anti-parallel coiled-coil that also has a proximal and a distal surface. Helix five (α5) connects the ring-shaped element to the anti-parallel coiled-coil, while helix eight (α8) is wrapped around the distal surface of the ring-shaped element. The inner surface of the hook-like structure is formed by the intersection of the proximal surface of the ring-shaped element with the proximal surface of the antiparallel coiled-coil.

In one embodiment, the N-terminal domain of the crystal comprises the amino acid sequence of Arg Xaa ^{H}Xaa Leu Xaa Xaa Trp ^{H}Xaa Glu Xaa Gln Xaa Trp (SEQ ID NO:1), where ^{H}Xaa can be either Ile, Leu, Val, Phe, or Tyr and Xaa can be any amino acid. In another embodiment, the crystal of the N-terminal domain of the STAT protein is contained in a STAT fragment that consists of 100 to 150 amino acids. In a preferred embodiment, the STAT fragment comprises amino acids 4-112 of SEQ ID NO:2. In a more preferred embodiment, the crystal contains an N-terminal domain of a STAT protein comprising amino acid residues 2-123 of SEQ ID NO:2 with 5 additional amino acid residues N-terminal to amino acid residue number 2, i.e., from the N-terminus GLY Ser Gly Gly Gly, amino acid residue 2. In one embodiment, the crystal effectively diffracts X-rays to allow the determination of the atomic coordinates of the N-terminus to a resolution of 1.45 Angstroms.

In a second aspect, the invention provides a dimerization interface of STAT N-domains, Interface II, deduced from the crystal structure provided by the invention, and shown in FIG. 1b, formed such that contact exists between amino acid residues Gln8 (Q8), Ile12 (I12), and Leu15 (L15) of α helices 1 and 2, Met28 (M28) and Glu29 (E29) of α helix 3 of a first STAT protein partner of the dimer, and Leu77 (L77) and Leu78 (L78) in α helix 7 of a second STAT protein partner of the dimer;

In a third aspect, the invention provides screening methods for identifying a compound capable of enhancing or inhibiting STAT-STAT dimeric interactions at Interface II. Identified agents include agonists, e.g., compounds capable of enhancing dimer-dimer interaction at Interface II, and antagonists, e.g., compounds capable of inhibiting dimer-dimer interactions at Interface II.

In one embodiment, a library of compounds is screened by assaying the binding activity of a STAT protein to its DNA binding site. This assay is based on the ability of the N-terminal domain of STAT proteins to substantially enhance the binding affinity of two adjacent STAT dimers to a pair of closely aligned DNA binding sites, i.e., binding sites separated by approximately 10 to 15 base pairs. Such compound libraries include phage libraries as described below, chemical libraries compiled by the major drug manufacturers, mixed libraries, and the like. Any of such compounds contained in the screened libraries are suitable for testing as a prospective drug in the assays described below, including in a high throughput assay based on the methods described below.

In a fourth aspect, the invention provides three-dimensional structural information for the design of small molecules capable of enhancing or inhibiting dimer-dimer interaction at Interface II. In one embodiment, virtual ligand docking and screening techniques are used to identify and/or design a compound capable of binding with high affinity to Interface II. Identified or designed compounds are then tested in in vitro and in vivo assays as described below to determine their ability to enhance or inhibit dimer-dimer interaction at Interface II.

In a fifth aspect, the invention also provides a method for identifying a compound capable of modulating the ability of adjacent STAT protein dimers to interact at Interface II and bind to adjacent DNA binding sites. In one embodiment, the agent is designed by rational drug design with the three-dimensional structure of Interface II. The binding affinity of the STAT protein (or of a fragment thereof that comprises the N-terminal domain) for a nucleic acid comprising two adjacent weak STAT DNA binding sites in the presence and absence of the test compound is determined. The binding affinity of the STAT protein (or the fragment) for a nucleic acid comprising a single strong STAT binding site in the presence and absence of the test compound is also determined. Next a comparison is made between the binding affinities of the STAT protein (or the fragment) is measured for the two adjacent weak STAT DNA binding sites in the presence and absence of the test compound with that determined for the STAT protein (or the fragment) for the single strong STAT binding site in the presence and absence of the test compound. A test compound which causes an increase in the binding affinity measured for the two adjacent weak STAT DNA binding sites but not in the binding affinity measured for the single strong STAT binding site is identified as a potential drug that enhances the interaction between adjacent activated STAT dimers. On the other hand, a test compound which causes a decrease in the binding affinity measured for the two adjacent weak STAT DNA binding sites but not in the binding affinity measured for the single strong STAT binding site is identified as a potential drug that inhibits the interaction between adjacent activated STAT dimers.

In a sixth aspect, the invention further provides a method for identifying a compound that enhances or diminishes the ability of STAT protein dimers to induce the expression of a gene operably under the control of a promoter containing at least two adjacent weak binding sites for STAT protein dimers. In one embodiment, the level of expression of a first reporter gene and a second reporter gene contained by a host cell in the presence and absence of the test compound is determined. The first reporter gene is operably linked to a first promoter containing at least two adjacent weak binding sites for STAT protein dimers, and the second reporter gene is operably linked to a second promoter comprising at least one strong binding site for a STAT protein dimer. The binding of STAT protein dimers to the two adjacent weak binding sites induces the expression of the first reporter gene, and the binding of the STAT protein dimer to the strong binding site induces the expression of the second reporter gene. In addition the host cell either naturally contains STAT protein dimers or is modified and/or induced to contain them. The level of expression of the first reporter gene is then compared with that of the second reporter gene in the presence and absence of the potential drug. When the presence of the potential drug results in an increase in the level of expression of the first reporter gene but not that of the second reporter gene, the test compound is identified as a potential drug that enhances the ability of STAT protein dimers to induce the expression of a gene operably under the control of a promoter containing at least two adjacent weak binding sites for STAT protein dimers. On the other hand, when the presence of a test compound results in a decrease in the level of expression of the first reporter gene but not that of the second reporter gene, the test compound is identified as a potential drug that inhibits the ability of STAT protein dimers to induce the expression of a gene operably under the control of a promoter containing at least two adjacent weak binding sites for STAT protein dimers.

In an alternative embodiment, the first reporter gene is contained by a first host cell, and the second reporter gene is contained by a second host cell. In this case, both the first host cell and second host cell contain STAT protein dimers. In one embodiment, the weak STAT binding sites are selected from sites present in the regulatory regions of the MIG gene, the c-fos gene, the interferon-γ gene. In a related embodiment, the strong STAT binding site is selected from the mutated cfos-promoter element, M67, the S1 site, and the IRF-1 gene promoter. In preferred embodiments, the host cell or host cells are mammalian cells.

Other objects and advantages will become apparent from a review of the ensuing detailed description taken in conjunction with the following illustrative drawing.

### BRIEF DESCRIPTION OF THE DRAWINGS

FIG. 1 shows close-up views of dimer Interface I (a) and Interface II (b), and the residues involved in dimer formation are indicated. The structures were drawn using Ribbons (Carson (1991) J. Appl. Cryst. 24:958), and the PDB coordinates 1BGF for the STAT4 N-domain (Vinkemeier et al. (1998) supra).

FIG. 2: Analytical ultracentrifugation sedimentation equilibrium data. Representative results for the wild type protein and some of the STAT1 N-domain mutant proteins are shown. In each case, the upper panel shows the residual difference between experimental and fitted values by its standard deviation, and the lower panel shows the equilibrium profile. The variance (V) between the fitted and experimental values, and calculated molecular mass (M) in daltons are indicated. The theoretical molecular weight of the STAT1 N-domain monomer is 15,223 da.

FIG. 3: Circular dichroism spectra of STAT1 N-domain proteins. The spectra for wild type STAT1 N-domain (blue), STAT1 F77A (green) and STAT1 L78A (red).

### DETAILED DESCRIPTION OF THE INVENTION

Before the present methods and compositions are described, it is to be understood that this invention is not limited to particular methods, compositions, and experimental conditions described, as such methods and compounds may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

Definitions

As used herein, the term “STAT” or “STAT protein” includes a particular family of transcription factor consisting of the Signal Transducers and Activators of Transcription proteins. Currently, there are seven STAT family members which have been identified, numbered STAT 1, 2, 3, 4, 5A, 5B, and 6. STAT proteins include proteins derived from alternative splice sites such as human STAT1α and STAT1β, i.e., STAT1β is a shorter protein than STAT1α and is translated from an alternatively spliced mRNA. Modified STAT proteins and functional fragments of STAT proteins are included in the present invention.

The “N-terminal domain” of a STAT protein is used interchangeably herein with the “N-terminal cooperative domain” and refers to the N-terminal portion of a STAT protein involved in STAT protein dimer-dimer interaction at a weak STAT DNA binding site. Preferably the amino acid of the N-terminal domain comprises SEQ ID NO:1. In one particular embodiment the STAT protein is STAT-4 comprising amino acids 2-123 of SEQ ID NO:2.

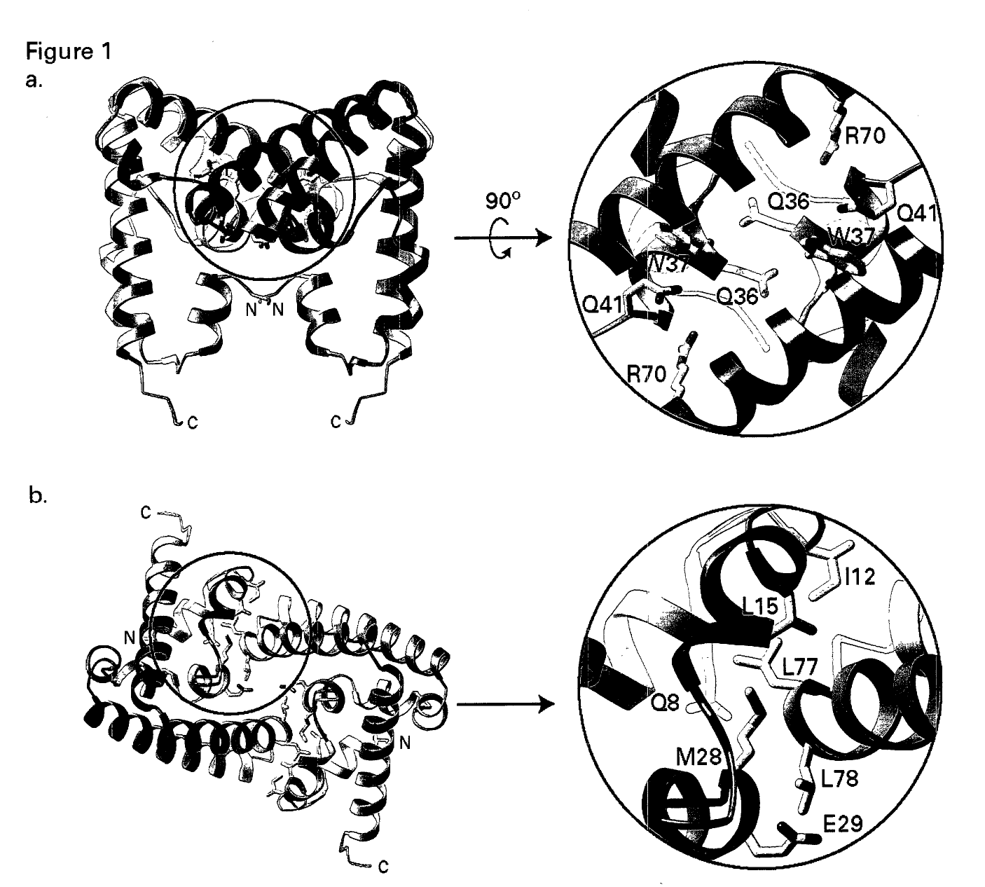

By the term “Interface I” is meant a region between two STAT molecules identified through analysis of the crystal structure of the N-domain of STAT4 (amino acid residues 1-124) involving amino acid residue Trp 37 (W37) shown in FIG. 1a.

By the term “Interface II” is meant a region between two STAT molecules identified through analysis of the crystal structure of the N-domain of STAT4 (amino acid residues 1-124), formed between amino acid residues Gln8 (Q8), Ile 12 (I12), and Leu15 (L15) of α helices 1 and 2, Met28 (M28) and Glu29 (E29) of α helix 3 of one partner of the dimer, and Leu77 (L77) and Leu78 (L78) in α helix 7 of the other partner of the dimer.

General Description

Earlier work on the crystal structure of the N-domain of STAT4 (residues 1-124) (Vinkemeier et al. (1998) supra) and of the core (residues ˜130-˜715; lacking the N-domain) STAT1 and STAT3β dimers bound to DNA (Becker et al. (1998.) Nature 394:145-151; Chen et al. (1998) Cell 93:827-839), led to the current understanding of the molecular architecture of STAT proteins. The N-domain of STAT is linked to the core via a flexible linker of ˜24 residues, and it was suggested that dimerization of the N-domains of adjacent STAT dimers on DNA leads to the formation of higher order STAT complexes on DNA (Chen et al. (1998) supra). The N-domain of STAT4, which is highly similar to STAT1 (51% sequence identity) was crystallized with one molecule in the asymmetric unit. Mutation of Trp 37, a residue located between two molecules at a crystal packing interface, led to the loss of cooperative STAT binding to tandem sites on DNA (Vinkemeier et al. (1998) supra; John et al. (1999) supra). Consequently, prior interpretations of structure and physiologically significant interactions were based in terms of that putative dimer interface seen in the crystal (Vinkemeier et al. (1998) supra).

The instant invention is based in part on the realization of a second interface domain of the crystal packing in the same crystal form, suggested to be relevant in solution. Crystal packing in the STAT4 crystal initially suggested one interface as potentially relevant for dimer formation. Interface I (FIG. 1a), originally analyzed by Vinkemeier et al. (1998) supra, is essentially polar, with 1,458 Å² of total surface area buried (calculated using a 1.4 Å probe radius). An alternate interface, termed “Interface II”, is more extensive (2,030 Å² total surface area buried), and contains hydrophobic residues (FIG. 1b).

As described below, point mutations in STAT1 were introduced at several sites at each of Interface I and II. The dimerization properties of these mutant proteins are shown in Table 1. The point mutation introduced in each of the STAT1 N-domain mutant proteins is indicated in the first column. The approximate molecular weight estimated by sedimentation equilibrium experiments, and migration as a monomer or dimer species on gel filtration analysis, is shown for each mutant protein.

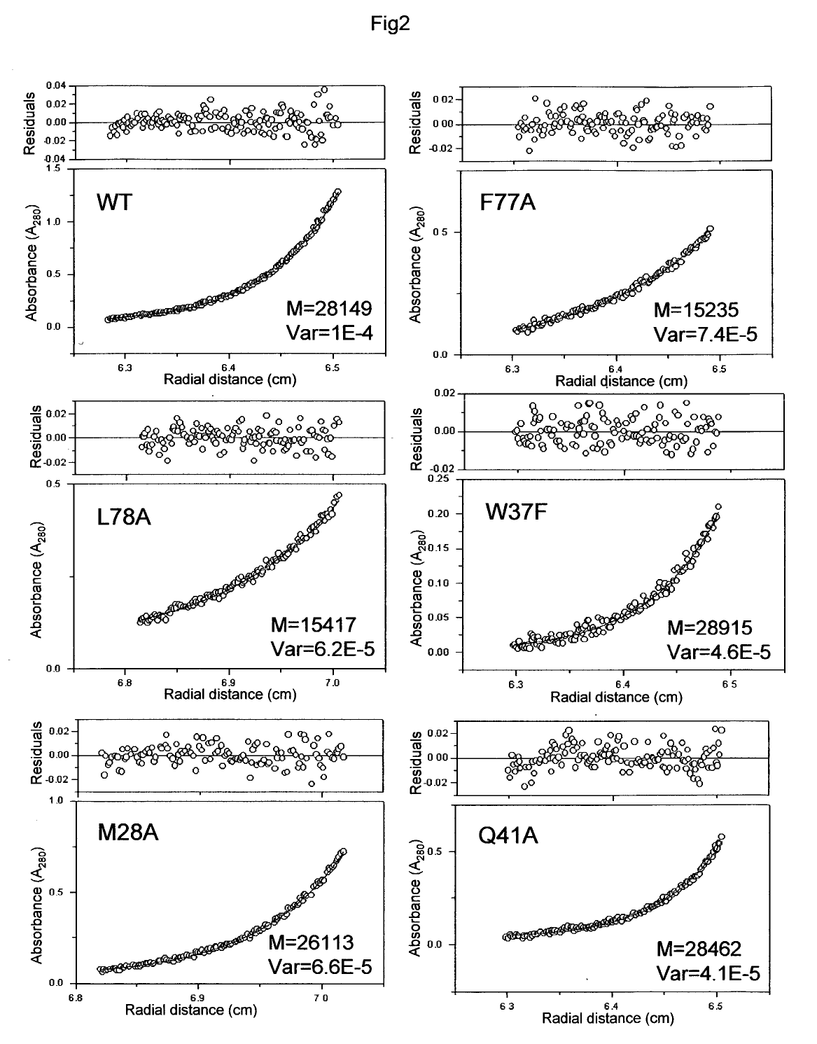

At interface I (Vinkemeier et al. (1998) supra) residues Trp37, Gln41, Gln36, and Arg70 were mutated to Ala. STAT1 (W37A) was expressed very poorly and we were unable to study the properties of this protein. A low level of expression of this mutant STAT protein has also been reported in another study (Murphy et al. (2000) Mol. Cell. Biol.20:7121-7131). The production of full length STAT1 (W37A) frequently leads to proteolytic degradation of the protein. However, sufficient amounts of the N-domain of STAT1 (W37F) were obtained and purified, and this protein was shown to be a dimer, as shown by analytical ultracentrifugation (FIG. 2) and gel filtration analysis (Table 1). Trp 37 was thought to mediate dimer formation by participating in direct and water-mediated hydrogen bonds, interactions that would be disrupted in the W37F mutant. The N-domain of STAT1 (W37F) is stable and is still a dimer, suggesting that W37 is not a part of the dimer interface. The fact that dimer formation is unimpeded in the W37F mutant suggests that the loss of tetramer formation on tandem sites on DNA seen for the full length STAT1 (W37A) mutant (Vinkemeier et al. (1988) supra) is not due to a specific disruption of the N-domain dimer interface. Three other residues implicated in dimer formation at interface I were mutated individually to Ala in STAT1, and the mutants are all dimeric (Table 1).

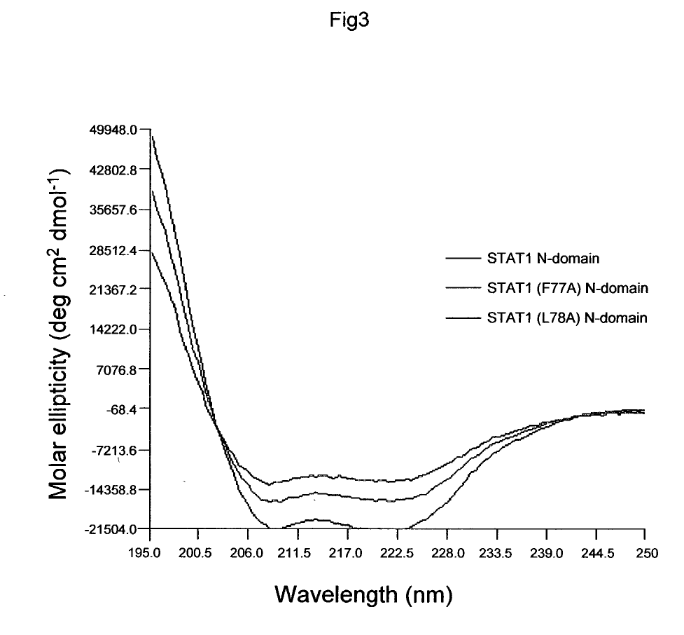

An alternate dimer interface determined by crystal packing (Interface II) is shown in FIG. 1b. Not only is Interface II more extensive than Interface I, it also involves interactions between hydrophobic residues (unlike the essentially polar nature of Interface I). Certain residues at interface II were individually replaced by Ala (Table 1) and the mutant STAT N-domains were examined for dimerization. Proteins containing mutations at one side of the interface, F77A and L78A, were monomers as seen by analytical ultracentrifugation (FIG. 2). To ensure that the mutant proteins (F77A and L78A) are folded properly, CD scans of these proteins were carried out as described below, and were found to be identical to wild type STAT1 N-domain (FIG. 3). The results for mutations at the other side of the interface provide evidence for interference with dimer formation. M28A migrated as an intermediate between dimer and monomer on gel filtration analysis, but appeared as a dimer by analytical ultracentrifugation analysis. S12A showed mainly aggregates and a small monomer population. The hydrophobic nature of the residues at positions 77 and 78 is conserved between STAT1 and STAT4, which has leucine residues at both positions. Likewise, Met 28 is conserved in STAT4.

These results indicate that Interface II is relevant to dimer formation in solution. In contrast to Interface I, for which none of the mutations introduced had a significant effect on dimer formation, several mutations at Interface II clearly interfered with the stability of the dimer.

A key conclusion that emerged from the previous analysis of the N-domain dimer was that the distance between the C-terminal residues in the dimer was consistent with the placement of the N-domain dimer between two adjacent STAT core dimers on tandem DNA sites (Chen et al. (1998) supra). This re-interpretation of the N-domain dimer interface does not alter that conclusion. The original N-domain dimer had its C-termini located 30 Å apart (Vinkemeier et al. (1998) supra). The original N-domain dimer could be positioned between two STAT core dimers modeled on adjacently located sites on DNA so that the C-terminal region of each N-domain monomer was located about 27 Å away from an N-terminal region of the adjacent STAT core dimer, to which it would be connected by a flexible 24 residue tether (Chen et al. (1998) supra). The C-terminal residues of the newly proposed dimer are located ˜64 Å apart. The increased span between the C-termini means that this dimer can be positioned between two adjacent STAT core dimers modeled on DNA with essentially no gap at the junction points.

Virtual Ligand Screening via Flexible Docking Technology

Current docking and screening methodologies can select small sets of likely lead candidate ligands from large libraries of compounds using a specific receptor structure. Such methods are described, for example, in Abagyan and Totrov (2001) Current Opinion Chemical Biology 5:375-382, herein specifically incorporated by reference in its entirety.

Virtual ligand screening (VLS) based on high-throughput flexible docking is useful for designing and identifying compounds able to bind to a specific receptor structure. VLS can be used to virtually sample a large number of chemical molecules without synthesizing and experimentally testing each one. Generally, the methods start with receptor modeling which uses a selected receptor structure derived by conventional means, e.g., X-ray crystallography, NMR, homology modeling. A set of compounds and/or molecular fragments are then docked into the selected binding site using any one of the existing docking programs, such as for example, MCDOCK (Liu et al. (1999) J. Comput. Aided Mol. Des. 13:435-451), SEED (Majeux et al. (1999) Proteins 37:88-105; DARWIN (Taylor et al. (2000) Proteins 41:173-191; MM (David et al. (2001) J. Comput. Aided Mol. Des. 15:157-171. Compounds are scored as ligands, and a list of candidate compounds predicted to possess the highest binding affinities generated for further in vitro and in vivo testing and/or chemical modification.

In one approach of VLS, molecules are “built” into a selected binding pocket prior to chemical generation. A large number of programs are designed to “grow” ligands atom-by-atom [see, for example, GENSTAR (Pearlman et al. L(1993) J. Comput. Chem. 14:1184), LEGEND (Nishibata et al. (1993) J. Med. Chem. 36:2921-2928), MCDNLG (Rotstein et al. (1993) J. Comput-Aided Mol. Des. 7:23-43), CONCEPTS (Gehlhaar et al. (1995) J. Med. Chem 38:466-472] or fragment-by-fragment [see, for example, GROUPBUILD (Rotsein et al. (1993) J. Med. Chem. 36:1700-1710), SPROUT (Gillet et al. (1993) J. Comput. Aided Mol. Des. 7:127-153), LUDI (Bohm (1992) J. Comput. Aided Mol. Des. 6:61-78), BUILDER (Roe (1995) J. Comput. Aided Mol. Des. 9:269-282), and SMOG (DeWitte et al. (1996) J. Am. Chem. Soc. 118:11733-11744].

Methods for scoring ligands for a particular receptor are known which allow discrimination between the small number of molecules able to bind the receptor structure and the large number of non-binders. See, for example, Agagyan et al. (2001) supra, for a report on the growing number of successful ligands identified via virtual ligand docking and screening methodologies.

The invention provides methods for identifying agents (e.g., candidate compounds or test compounds) that bind with high affinity to the dimer-dimer interface domain termed Interface II. Agents identified by the screening method of the invention are useful as candidate therapeutics.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

Binding Assays for Drug Screening Assays

The drug screening assays of the present invention may use any of a number of assays for measuring the stability of a STAT-STAT dimeric interaction, including N-terminal dimeric STAT fragments and/or a dimeric STAT-STAT-DNA binding interaction. In one embodiment, the stability of a preformed DNA-protein complex between a dimeric STAT protein and its corresponding DNA binding site is examined as follows: the formation of a complex between the STAT protein and a labeled oligonucleotide is allowed to occur and unlabelled oligonucleotides are added in vast molar excess after the reaction reaches equilibrium. At various times after the addition of unlabelled competitor DNA, aliquots are layered on a running native polyacrylamide gel to determine free and bound oligonucleotides. In one preferred embodiment, the protein is STAT1α, and two different labeled DNAs are used, the natural cfos site, an example of a “weak” site, and the mutated cfos-promoter element, the M67 site (Wagner et al. (1990) EMBO J. 9:4477) an example of a “strong” site as described below. Other examples of weak sites include those in the promoter of the MIG gene, and those in the regulatory region of the interferon-γ gene. Other examples of strong sites include those such as the selected optimum site, S1 (Horvath et al. (1995) Genes & Devel. 9:984) or the promoter of the IRF-1 gene.

In a related binding assay, a nucleic acid containing a weak STAT binding site is placed on or coated onto a solid support. Methods for placing the nucleic acid on the solid support are well known in the art and include such things as linking biotin to the nucleic acid and linking avidin to the solid support. Dimeric STAT proteins are allowed to equilibrate with the nucleic acid and drugs are tested to see if they disrupt or enhance the binding. Disruption leads to either a faster release of the STAT protein which may be expressed as a faster off time, and or a greater concentration of released STAT dimer. Enhancement leads to either a slower release of the STAT protein which may be expressed as a slower off time, and/or a lower concentration of released STAT protein.

The STAT protein may be labeled as described below. For example, in one embodiment radiolabeled STAT proteins are used to measure the effect of a drug on binding. In another embodiment the natural ultraviolet absorbance of the STAT protein is used. In yet another embodiment, a Biocore chip (Pharmacia) coated with the nucleic acid is used and the change in surface conductivity can be measured.

In yet another embodiment, the affect of a test compound on interactions between N-terminal domains of STATs is assayed in living cells that contain or can be induced to contain activated STAT proteins, i.e., STAT protein dimers. Cells containing a reporter gene, such as the heterologous gene for luciferase, green fluorescent protein, chloramphenicol acetyl transferase or β-galactosidase, operably linked to a promoter comprising two weak STAT binding sites are contacted with a prospective drug in the presence of a cytokine which activates the STAT(s) of interest. The amount (and/or activity) of reporter produced in the absence and presence of the test compound is determined and compared. Test compounds which reduce the amount (and/or activity) of reporter produced are candidate antagonists of the N-terminal interaction, whereas test compounds which increase the amount (and/or activity) of reporter produced are candidate agonists. Cells containing a reporter gene operably linked to a promoter comprising strong STAT binding sites are then contacted with these test compounds, in the presence of a cytokine which activates the STAT(s) of interest. The amount (and/or activity) of reporter produced in the presence and absence of the test compound is determined and compared. Compounds which disrupt interactions between dimeric N-terminal domains of the STATs will not reduce reporter activity in this second step. Similarly, compounds which enhance interactions between dimeric N-terminal domains of STATs will not increase reporter activity in this second step.

In an analogous embodiment, two reporter genes each operably under the control of one or the other of the two types promoters described above can be comprised in a single host cell as long as the expression of the two reporter gene products can be distinguished. For example, different modified forms of green fluorescent protein can be used as described in U.S. Pat. No. 5,625,048, hereby incorporated by reference in its entirety.

Although cells that naturally encode the STAT proteins may be used, preferably a cell is used that is transfected with a plasmid encoding the STAT protein. For example transient transfections can be performed with 50% confluent U3A cells using the calcium phosphate method as instructed by the manufacturer (Stratagene). In addition as mentioned above, the cells can also be modified to contain one or more reporter genes, a heterologous gene encoding a reporter such as luciferase, green fluorescent protein or derivative thereof, chloramphenicol acetyl transferase, β-galactosidase, etc. Such reporter genes can individually be operably linked to promoters comprising two weak STAT binding sites and/or a promoter comprising a strong STAT binding site. Assays for detecting the reporter gene products are readily available in the literature. For example, luciferase assays can be performed according to the manufacturer's protocol (Promega), and β-galactosidase assays can be performed as described by Ausubel et al. (1994) in Current Protocols in Molecular Biology, J. Wiley & Sons, Inc.).

In one example, the transfection reaction can comprise the transfection of a cell with a plasmid modified to contain a STAT protein, such as a pcDNA3 plasmid (Invitrogen), a reporter plasmid that contains a first reporter gene, and a reporter plasmid that contains a second reporter gene. Although the preparation of such plasmids is now routine in the art, many appropriate plasmids are commercially available e.g., a plasmid with β-galactosidase is available from Stratagene.

The reporter plasmids can contain specific restriction sites in which an enhancer element having a strong STAT binding site or alternatively two tandemly arranged “weak” STAT binding sites can be inserted. In one particular embodiment, thirty-six hours after transfection of the cells with a plasmid encoding STAT-1, the cells are treated with 5 ng/ml interferon-γ Amgen for ten hours. Protein expression and tyrosine phosphorylation (to monitor STAT activation) can be determined by e.g., gel shift experiments with whole cell extracts.

Labels

Suitable labels include enzymes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the test and control marker gene.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. U.V. light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Engvall (1980) Methods in Enzymology 70:419-439 and in U.S. Pat. No. 4,857,453. Suitable enzymes include, but are not limited to, alkaline phosphatase, β-galactosidase, green fluorescent protein and its derivatives, luciferase, and horseradish peroxidase. Other labels for use in the invention include magnetic beads or magnetic resonance imaging labels.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Materials and Methods

The N-domain of human STAT1 (amino acid residues 1 to 124) was cloned as a C-terminal fusion to glutathione S-transferase (GST), in a pGEX2T vector (Amersham Biosciences) that had been modified to replace the thrombin protease cleavage site with a cleavage site for tobacco etch virus (TEV) protease (U.S. Pat. No. 6,312,887 B1). Site-directed mutagenesis was carried out using the Quikchange method (Stratagene). The construct and mutations were confirmed by sequencing.

The constructs were expressed in the E. coli strain BL21(λDE3). Cells were resuspended in buffer A (50 mM Tris pH 8.0, 150 mM NaCl and 1 mM DTT) and lysed in a French press. The lysate was clarified by high-speed centrifugation and the supernatant fraction was purified on a glutathione sepharose column on the Amersham Biosciences AKTA FPLC system. After washing the column with five column volumes of buffer A, the fusion protein was eluted using 20 mM reduced glutathione in buffer A. TEV protease was added to the pooled fractions and the digestion was carried out at 15° C. overnight. The N-domain and GST were separated on a HiTrap Q column (Amersham Biosciences), in buffer A using a 0-70% gradient of buffer B (50 mM Tris pH 8.0, 800 mM NaCl and 1 mM DTT) over 30 column volumes. The pooled fractions of the peak containing the STAT1 N-domain were concentrated and passed over a Superdex 75 column to separate any remaining GST, which migrates as a dimer of about 52 kDa. In the case of mutant proteins F77A and L78A, there was very poor separation between GST and STAT1 N-domain on a Q column. These proteins were well separated from GST on a Superdex 75 column.

For gel filtration analysis, 1.5 mg of purified STAT N domain protein in a volume of 500 μl was run on a 120 ml Superdex 75 column at a flow rate of 0.5 ml/min, in 50 mM Tris, pH 8.0, 100 mM NaCl and 1 mM DTT. Equilibrium sedimentation experiments were performed using a Beckman Optima XL-A analytical ultracentrifuge with an An-60 Ti rotor and six-sector cells. STAT N-domain proteins at concentrations of 0.65, 0.32 and 0.16 mg/ml were centrifuged in the gel filtration buffer, at 25,000 rev/min. at 4° C. for 20 h. Subsequently, absorbance measurements at 280 nm were taken in 0.001 cm radial steps and equilibrium was ascertained by comparing scans taken at 1 h intervals. The Optima XL-A/XL-I data analysis software from Beckman Coulter was used for data processing and curve fitting. A partial specific volume of 0.73 cm³/g was used and background absorbance was corrected empirically by allowing the baseline to float during the fitting calculations.

CD measurements were performed on an Aviv Model 215 Circular Dichroism Spectrometer at 25° C. using a 0.02 cm pathlength cuvette. The purified proteins were dialysed against PBS (10 mM sodium phosphate buffer, pH 7.4, 140 mM NaCl, 10 mM KCl) and diluted to a concentration of 40 μM. Spectra were recorded from 250 to 190 nm using a step of 0.5 nm and an averaging time of four seconds.

TABLE 1
Properties of the wild type and mutant STAT1 N-domain proteins

| | Sedimentation | Gel |
|---|---|---|
| STAT1 | equilibrium | filtration |
| Wild type | 28 kDa | dimer |
| Interface I | | |
| W37A | — | — |
| W37F | 28 kDa | dimer |
| Q41A | 27 kDa | dimer |
| Q36A | 29 kDa | dimer |
| R70A | 27 kDa | dimer |
| Interface II | | |
| Q8A | 27 kDa | dimer |
| S12A | 17 kDa + | not |
| | aggregates | examined |
| L15A | 28 kDa | dimer |
| M28A | 26 kDa | monomer |
| E29A | 27 kDa | dimer |
| F77A | 15 kDa | monomer |
| L78A | 15 kDa | not examined |

1. A method of identifying a compound capable of enhancing or inhibiting binding between Signal Transducer and Activator of Transcription (STAT) protein dimers to each other at an interface domain and/or a nucleic acid binding site, comprising:
(a) obtaining a set of atomic coordinates defining the three dimensional structure of a crystal of an N-terminal fragment of a STAT protein that effectively diffracts X-rays for the determination of the atomic coordinates of the N-terminal fragment to a resolution of 1.45 Å, wherein the N-terminal fragment of a STAT protein comprises amino acid residues 1-130 of SEQ ID NO:1, the crystal has a space group of P6₅22 and a unit cell of dimensions a=79.51 Å, b=79.51 Å, and c=84.68 Å, and wherein the interface domain is formed such that contact exists between amino acid residues Gln8 (Q8), Ile12 (I12), and Leu15 (L15) of α helices 1 and 2, Met28 (M28) and Glu29 (E29) of α helix 3 of a first STAT protein partner of the dimer, and Leu77 (L77) and Leu78 (L78) in α helix 7 of a second STAT protein partner of the dimer;
(b) contacting a test compound with two or more dimeric STAT proteins in the presence of a nucleic acid containing at least two adjacent binding sites for STAT protein dimers; and
(c) detecting the effect of the test compound on the binding of the dimeric STAT proteins to each other and/or to the nucleic acid binding site, wherein the test compound is identified as capable of enhancing or inhibiting binding between dimeric STAT proteins when it either enhances or inhibits the binding of dimeric STAT proteins to each other and/or the nucleic acid binding site.

2. The method of claim 1, wherein a test compound is a compound designed to bind the interface domain formed between amino acid residues Gln8 (Q8), Ile12 (I12), and Leu15 (L15) of α helices 1 and 2, Met28 (M28) and Glu29 (E29) of α helix 3 of a first STAT protein partner of the dimer, and Leu77 (L77) and Leu78 (L78) in α helix 7 of a second STAT protein partner of the dimer.

3. A method of identifying a compound capable of modulating binding between dimeric Signal Transducer and Activator of Transcription (STAT) proteins to each other at an interface domain and/or a nucleic acid binding site, comprising:
(a) obtaining a set of atomic coordinates defining the three dimensional structure of a crystal of an N-terminal fragment of a STAT protein that effectively diffracts X-rays for the determination of the atomic coordinates of the N-terminal fragment to a resolution of 1.45 Å, wherein the N-terminal fragment of a STAT protein comprises amino acid residues 1-130 of SEQ ID NO:1, the crystal has a space group of P6₅22 and a unit cell of dimensions a=79.51 Å, b=79.51 Å, and c=84.68 Å, and wherein the interface domain is formed such that contact exists between amino acid residues Gln8 (Q8), Ile12 (I12), and Leu15 (L15) of α helices 1 and 2, Met28 (M28) and Glu29 (E29) of α helix 3 of a first STAT protein partner of the dimer, and Leu77 (L77) and Leu78 (L78) in α helix 7 of a second STAT protein partner of the dimer;
(b) contacting a test compound with two or more dimeric STAT proteins in the presence of a nucleic acid containing at least two adjacent binding sites for STAT protein dimers; and
(c) detecting the effect of the test compound on the binding of the dimeric STAT proteins to each other and/or to the nucleic acid binding site, wherein the test compound is identified as capable of modulating binding between dimeric STAT proteins when the binding of dimeric STAT proteins to each other and/or the nucleic acid binding site is changed in the presence of the test compound compared to binding in the absence of the test compound.

4. The method of claim 1, wherein a test compound is a compound designed to bind the interface domain formed between amino acid residues Gln8 (Q8), Ile12 (I12), and Leu15 (L15) of α helices 1 and 2, Met28 (M28) and Glu29 (E29) of α helix 3 of a first STAT protein partner of the dimer, and Leu77 (L77) and Leu78 (L78) in α helix 7 of a second STAT protein partner of the dimer.

5. A method for identifying a compound that enhances or diminishes the ability of dimeric Signal Transducer and Activator of Transcription (STAT) proteins to induce the expression of a gene operably under the control of a promoter containing at least two adjacent weak binding sites for STAT protein dimers, comprising:
(a) obtaining a set of atomic coordinates defining the three dimensional structure of a crystal of an N-terminal fragment of a STAT protein that effectively diffracts X-rays for the determination of the atomic coordinates of the N-terminal fragment to a resolution of 1.45 Å, wherein the N-terminal fragment of a STAT protein comprises amino acid residues 1-130 of SEQ ID NO:1, the crystal has a space group of P6₅22 and a unit cell of dimensions a=79.51 Å, b=79.51 Å, and c=84.68 Å, and wherein the interface domain is formed such that contact exists between amino acid residues Gln8 (Q8), Ile12 (I12), and Leu15 (L15) of α helices 1 and 2, Met28 (M28) and Glu29 (E29) of α helix 3 of a first STAT protein partner of the dimer, and Leu77 (L77) and Leu78 (L78) in α helix 7 of a second STAT protein partner of the dimer;
(b) measuring the level of expression of a first reporter gene and a second reporter gene contained by a host cell in the presence and absence of a test compound, wherein the first reporter gene is operably linked to a first promoter containing at least two adjacent weak binding sites for STAT protein dimers, and the second reporter gene is operably linked to a second promoter comprising at least one strong binding site for a STAT protein dimer, and wherein the binding of STAT protein dimers to the two adjacent weak binding sites induces the expression of the first reporter gene, and the binding of the STAT protein dimer to the strong binding site induces the expression of the second reporter gene, and wherein the host cell contains STAT protein dimers; and
(c) comparing the level of expression of the first report gene with that of the second reporter gene in the presence and absence of the test compound, wherein when the presence of the test compound results in an increase in the level of expression of the first reporter gene but not that of the second reporter gene, the test compound is identified as a compound that enhances the ability of STAT protein dimers to induce the expression of a gene operably under the control of a promoter containing at least two adjacent weak binding sites for STAT protein dimers, and when the presence of a test compound results in a decrease in the level of expression of the first reporter gene but not that of the second reporter gene, the test compound is identified as a compound that inhibits the ability of STAT protein dimers to induce the expression of a gene operably under the control of a promoter containing at least two adjacent weak binding sites for STAT protein dimers.

8. The method of claim 7, wherein a test compound is a compound designed to bind the interface domain formed between amino acid residues Gln8 (Q8), Ile12 (I12), and Leu15 (L15) of α helices 1 and 2, Met28 (M28) and Glu29 (E29) of α helix 3 of a first STAT protein partner of the dimer, and Leu77 (L77) and Leu78 (L78) in α helix 7 of a second STAT protein partner of the dimer.

9. The method of claim 7, wherein the host cells is a mammalian cell.

10. The method of claim 7, wherein the first reporter gen is contained by a first host cell, and the second reporter gene is contained by a second host cell, and wherein the first and second host cells both contain STAT protein dimers.

11. The method of claim 7, wherein the weak STAT binding sites are selected from the group consisting of binding sites present in the regulatory regions of the MIG gene, the c-fos gene, and the interferon-γ gene.
